(19) United States
(12) Patent Application Publication
(10) Pub. No.: US 2013/0030389 A1
(43) Pub. Date: Jan. 31, 2013

(54) METHOD OF INSERT MOLDING A CONNECTION FOR CATHETER WITH VARYING DIAMETERS

(71) Applicant: Gerard M. HAMILTON, Limerick (IE)

(72) Inventor: Gerard M. HAMILTON, Limerick (IE)

(73) Assignee: Teleflex Medical Incorporated, Durham, NC (US)

(21) Appl. No.: 13/559,254

(22) Filed: Jul. 26, 2012

Related U.S. Application Data
(60) Provisional application No. 61/512,339, filed on Jul. 27, 2011.

(51) Int. Cl. B29C 65/70; A61M 25/14

(52) U.S. Cl. 604/264; 264/263

(57) ABSTRACT

The present invention provides in some embodiments, a method for insert molding a coupling between catheter segments with different diameters and/or a catheter having different diameters. Generally, the method can include the steps of providing first and second tubes having larger and smaller diameters, respectively and positioning the first and second tubes onto a core pin assembly having an outer surface at one section which forms a contoured inner profile for a joint section to join the first and second tubes. Additionally, the method can include injection molding the joint section to join the first and second tubes, to form the catheter tube with the contoured inner profile at the joint section.

### CROSS-REFERENCE TO RELATED APPLICATION

This application claims priority to, and is a non-provisional of, pending provisional U.S. patent application entitled, “Method of Insert Molding a Connection for Catheter with Varying Diameters”, filed Jul. 27, 2011, having a Ser. No. 61/512,339, the disclosure of which is hereby incorporated by reference in its entirety.

### FIELD OF THE INVENTION

The present invention generally relates to the formation of catheters. More particularly, the present invention pertains to an method of insert molding a connection for catheters with varying diameters.

### BACKGROUND OF THE INVENTION

In the catheter area of medical devices it is increasingly desirable to have a catheter having sections of different diameters. Using currently available manufacturing techniques it is quite challenging and therefore, costly to manufacture a catheter having a larger diameter portion and a smaller diameter portion or sections having significantly different inner diameters.

One currently known method for joining two catheter sections having different diameters is to hand assemble the segments of catheter having varying diameters over a fluoropolymer liner. However, this method of assembly becomes difficult after the ratio between the large and small diameters exceeds 1.1, because the fluoropolymer liner can only be stretched very slightly before the ability to make a laminated composite catheter is compromised.

Another currently known method for creating a catheter having segments with significantly different inner diameters is to flare an extrusion from one diameter to a larger diameter. This method also presents problems, which include a limited length of the flare section, cosmetic issues and the requirements for relatively thin walls on the base extrusion that is to be flared.

It is therefore desirable to provide a method for joining the catheter tubes that provides a structurally stable end product and is less costly.

### SUMMARY OF THE INVENTION

The foregoing needs are met, to a great extent, by the present invention, wherein in some embodiments a method of insert molding a connection for catheters with varying diameters that is capable of overcoming the disadvantages described herein at least to some extent is provided.

In accordance with an embodiment of the present invention a method of forming a medical catheter tube having sections of different diameter, can include providing first and second tubes having larger and smaller diameters, respectively. The method can also include positioning the first and second tubes onto a core pin assembly having an outer surface at one section which forms a contoured inner profile for a joint section to join the first and second tubes. Additionally, the method can include injection molding the joint section to join the first and second tubes, to form the catheter tube with the contoured inner profile at the joint section. In some embodiments the injection molding of the joint section can include a plurality of injection molding steps to form a plurality of layers of the joint section.

In accordance with another embodiment of the present invention, a method of forming a medical catheter tube having sections of different diameter coupled at a joint section, can include providing a first length of tubing having a first, larger diameter and providing a second length of tubing having a second, smaller diameter. The method can include providing a set of one or more core pins arranged to provide a pre-determined inner surface profile of the catheter tube at the joint section, and positioning the first length of tubing over the set of core pins, to form a first sub-assembly. The method can also include placing the first sub-assembly into a clamshell fixture which holds the first length of tubing fixed to the set of core pins and leaves a first end portion of the set of core pins exposed, to form a second sub-assembly.

Additional steps can include placing the second sub-assembly into a mold base, and positioning the second length of tubing over the first end portion of the set of core pins, leaving a section of the set of core pins exposed. The method can also include placing a cover mold portion over the mold base to form an inner space around the exposed section of the set of core pins.

Additionally, the method can include injection molding the joint section of the catheter tube by injecting a material into the inner space, said joint section coupling the first and second lengths of tubing and providing the pre-determined inner surface profile of the catheter tube at the joint section.

In accordance with still another aspect of the present invention, the method of can include detaching the cover mold portion from the mold base and removing a third sub-assembly which includes the finished catheter tube, clamshell fixture and set of core pins. The method can also include placing the third sub-assembly into a catheter removal fixture to hold the third sub-assembly. Further the method can include, removing the set of core pins from the third sub-assembly, and opening the clamshell fixture to remove the finished catheter tube.

In accordance with another aspect of the present invention, the method can include injection molding of the joint section of the catheter tube comprises a plurality of injection molding steps to form a plurality of layers of the joint section. Additionally, the set of core pins can be expandable. The core pins can also interlock with each other.

In accordance with another aspect of some embodiments of the invention, a catheter is provided. The catheter may include: a first section having a first diameter, a second section having a second diameter greater than the first diameter; and a joint section connecting the first section and the second sections, the joint section having a tapered portion tapering the inner diameter of the molded section down from the first diameter to the second diameter and the joint section is molded to the first section and the second section.

There has thus been outlined, rather broadly, certain embodiments of the invention in order that the detailed description thereof, herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 illustrates a diagram of the steps of a method in accordance with an embodiment of the invention.

FIG. 2 illustrates a schematic diagram of a core pin assembly inside the two catheter shafts of different diameter, as well as the space defined to form the coupling between the large diameter catheter shaft and the small diameter catheter shaft in accordance with the method of the invention.

FIG. 3 illustrates a clam shell assembly with core pins in accordance with the invention.

FIG. 4 illustrates a sectional view of the clam shell assembly illustrated in FIG. 3 in accordance with the invention.

FIG. 5 illustrates an end view of the clam shell assembly illustrated in FIGS. 3 and 4.

FIG. 6 illustrates a catheter having segments with different diameters as joined in accordance with an embodiment of the invention.

FIG. 7 illustrates an exploded view of the catheter illustrated in FIG. 6.

FIG. 8 illustrates a mold assembly for forming the connection between a large diameter shaft and a small diameter shaft in accordance with an embodiment of the invention.

FIG. 9 illustrates a core pin assembly in accordance with an embodiment of the invention.

### DETAILED DESCRIPTION

The present invention provides in some embodiments, a method for insert molding a coupling between catheter segments with different diameters. Generally, the method can include the steps of providing first and second tubes having larger and smaller diameters, respectively, and positioning the first and second tubes onto a core pin assembly having an outer surface at one section which forms a contoured inner profile for a joint section to join the first and second tubes. Additionally, the method can include injection molding the joint section to join the first and second tubes, to form the catheter tube with the contoured inner profile at the joint section.

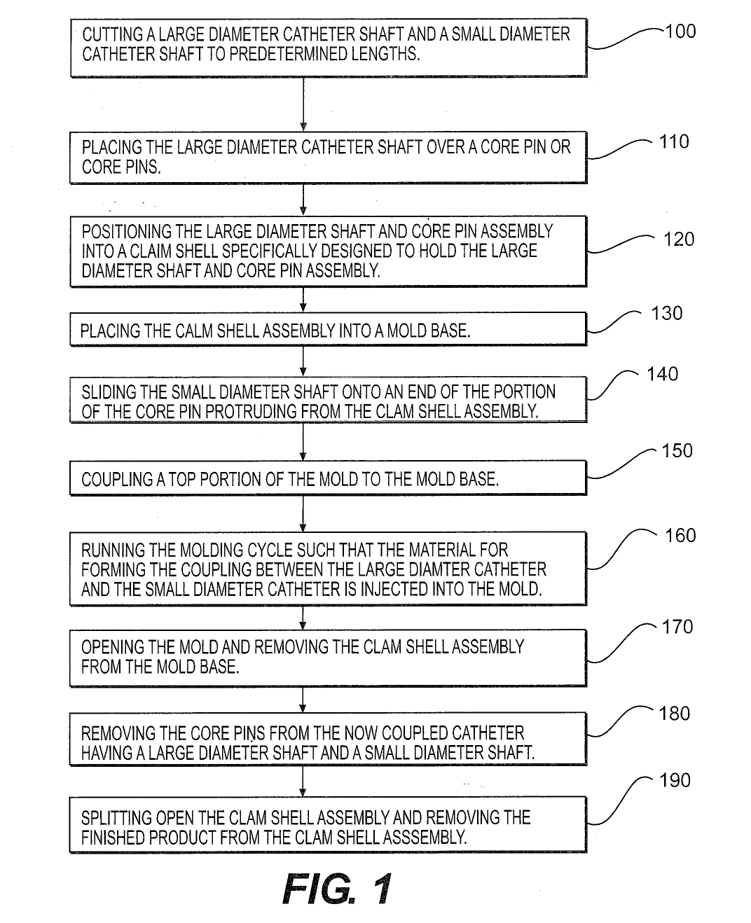

The invention will now be described with reference to the drawing figures, in which like reference numerals refer to like parts throughout. In a first aspect of some embodiments of the invention, FIG. 1 illustrates a method for insert molding a coupling between catheter segments with different diameters. The method can include step 100 of cutting catheter tubing including a large diameter catheter shaft and a small diameter catheter shaft to lengths requested by a customer. The large and small diameter catheters can take the form of any catheter suitable for joining with an injection molded coupling. More specifically, the catheter can take the form of reinforced or non-reinforced tubing. If reinforced tubing is used the tube can be reinforced with braid or coil or any other suitable form of reinforcement.

Step 110 includes placing the large diameter catheter shaft over a core pin or set of core pins. The core pin can take the form of a single pin or alternately can be formed of multiple core pins that take the shape of the internal structure of the coupling between the large diameter shaft and the small diameter shaft. The outer surface of the core pin is therefore configured to provide a pre-determined inner profile, such as a contour or a flared coupling, between the large diameter shaft and the small diameter shaft, such that the coupling increases in diameter from the small diameter shaft to the large diameter shaft. The core pin assembly may also be configured to allow a small amount of the molding material to coat a portion of an inside surface of either shaft, and, in one embodiment, the large diameter shaft.

Step 120 can include positioning the large diameter shaft and core pin assembly into a clam shell specifically designed to hold the large diameter shaft and core pin assembly. The clam shell assembly can be designed such that it encapsulates the entirety of the large diameter shaft. A portion of the core pin assembly will extend beyond an end of the clam shell and remain exposed. This portion of the core pin includes the structure for forming the interior surface of the coupling, or joint section of the catheter, between the large diameter shaft and the small diameter shaft and also structure for holding the small diameter shaft.

The method can also include step 130 of placing the clam shell assembly into a mold base. The mold base may define a bottom half of the outer surface of the coupling or joint section between the large diameter shaft and the small diameter shaft. Step 140 can include sliding the small diameter shaft onto an end of the portion of the core pin assembly protruding from the clam shell assembly. A distal portion of the small diameter shaft can extend beyond an end of the mold base. Step 150 includes coupling a top or cover portion of the mold to the mold base. The top portion of the mold may also define a top half of the outer surface of the coupling between the large diameter shaft and the small diameter shaft. The two mold halves may thereby define, together with the exposed portion of the core pin assembly, an inner space for injection molding the joint or coupling between the two catheter shafts. It should be noted that the top and bottom halves of the mold can also define a space such that a thin coat of the material for the coupling is applied to an outer surface of either shaft, and in one embodiment, a proximal end of the small diameter shaft.

Step 160 can include running the molding cycle such that the material for forming the coupling between the large diameter catheter shaft and the small diameter catheter shaft is injected into the mold. The material used to form the coupling can be any suitable polymer that is compatible with the material of the large diameter and small diameter catheter segments. Therefore, the material used may differ based on the material of customer's requested large and small diameter catheters. The method can also include step 170 of opening the mold and removing the clam shell assembly from the mold base. Step 180 can include removing the core pins from the now finished catheter having a large diameter shaft and a small diameter shaft coupled by the injection molded joint section, as described herein. The core pins can be removed manually or using any suitable fixture or device to remove them. In one embodiment, the fixture used to remove the core pins can include a clamp device that is able to attach to and lift a top plate of the clam shell. The method can also include step 190 of splitting open the clam shell assembly and removing the finished product from the clam shell assembly.

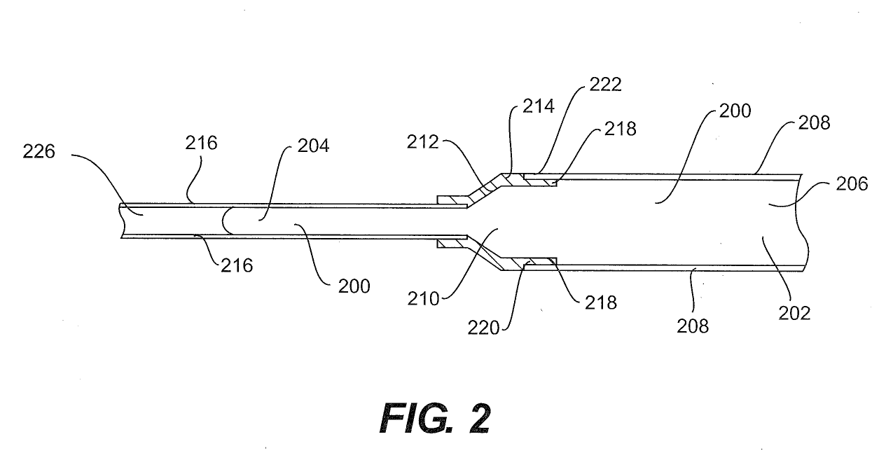

FIG. 2 illustrates a schematic diagram of a section of a core pin assembly with catheter shafts positioned, as well as the space defined to form the coupling between sections of the large diameter catheter shaft and the small diameter catheter shaft in accordance with the method of the invention. As illustrated in FIG. 2, a core pin assembly 200 has a proximal end 202 and a distal end 204. The core pin assembly 200 can be formed from a single pin tooled to have the desired shape and dimensions or can be formed from multiple expandable or interlocking pins or segments in order to obtain the desired shape and dimensions. The proximal end 202 of the core pin assembly 200 is configured to fit within a lumen 206 of the large diameter catheter shaft 208. The core pin assembly 200 can be configured to extend through the entirety of the length of the lumen 206 of the large diameter catheter 208.

FIG. 2 also illustrates that a middle portion 210 of the core pin assembly 200 can be formed to take the shape of an interior surface 212 of a coupling 214 to be formed between the large diameter catheter shaft 208 and the small diameter catheter shaft 216. In FIG. 2, the coupling 214 is illustrated as shaded portion 214. The middle portion 210 of the core pin assembly can also define an indented portion 218 disposed under a distal end 220 of the large diameter catheter shaft 208. The indented portion 218 allows a small amount of the material used to form the coupling 214 to flow between the large diameter shaft 208 and the core pin assembly, such that a thin film of the material used to form the coupling 214 also coats a portion of an interior surface 222 of the end of the large diameter catheter shaft 208. The distal portion 204 of the core pin assembly 200 is configured to fit within a lumen 226 of the small diameter catheter shaft 216. The distal portion 204 of the core pin assembly 200 can extend through all or a portion of the small diameter catheter shaft 216.

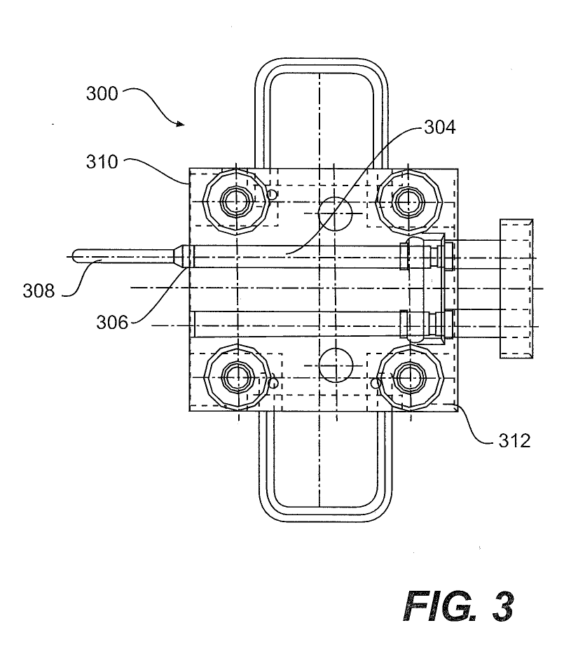

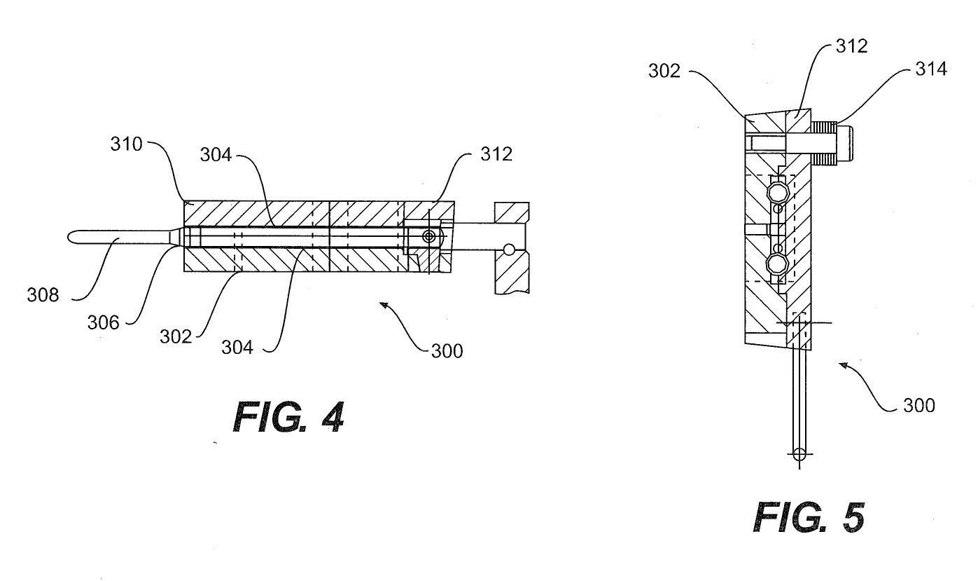

FIGS. 3, 4, and 5 illustrate a clam shell assembly 300 in accordance with an embodiment of the invention. FIG. 3 illustrates the clam shell 300 in its assembled form. A bottom half 302 and a top half 312 of the clam shell 300 are specially designed to encapsulate the entire large diameter shaft. For instance, in this embodiment, the large diameter shaft can sit within a tubular indentation 304 formed in the clam shell assembly. The clam shell 300 is also designed to allow a middle portion 306 and an outer portion 308 of the inner core pins to protrude beyond an edge 310 of the clam shell 300. The bottom half 302 and the top half 312 of the clam shell 300 can be spring loaded closed using spring 314, as illustrated in FIG. 5.

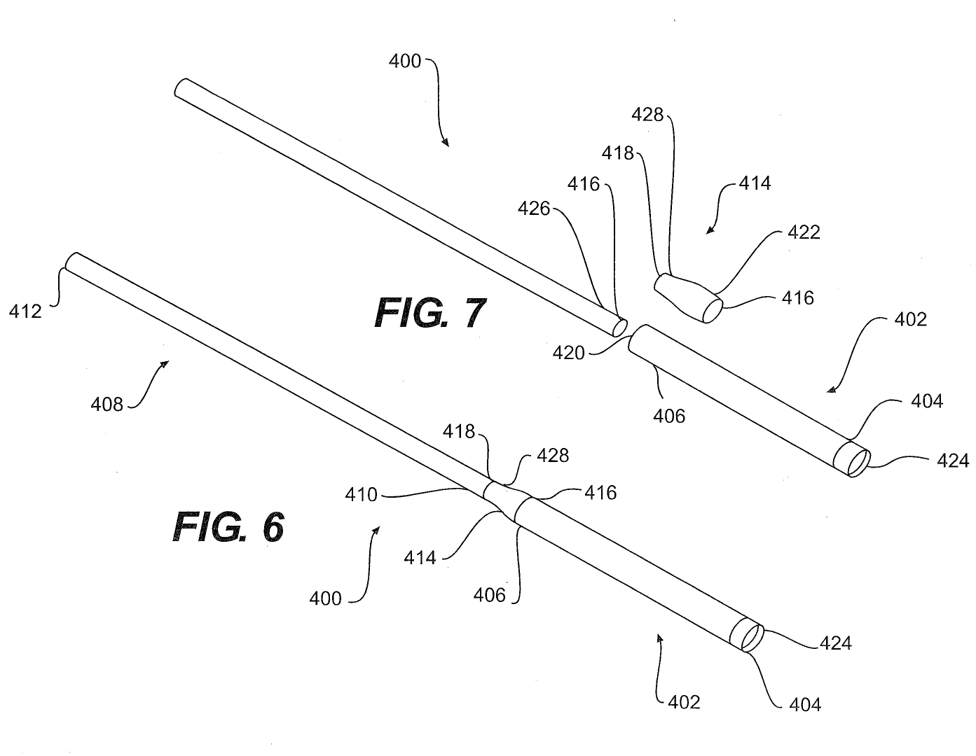

FIG. 6 illustrates one example of a finished catheter 400 having the large diameter shaft and the small diameter shaft coupled together using the injection molding method described above. FIG. 7 illustrates an exploded view of the catheter 400 illustrated in FIG. 6. FIGS. 6 and 7 show the large diameter shaft 402 having a proximal end 404 and a distal end 406 and the small diameter shaft 408 having a proximal end 410 and a distal end 412. The large and small diameter catheters can take the form of any catheter suitable for joining with an injection molded coupling. More specifically, the catheter can take the form of reinforced or non-reinforced tubing. If reinforced tubing is used the tube can be reinforced with braid or coil or any other suitable form of reinforcement. The large diameter shaft 402 and the small diameter shaft 408 are connected by injection molded coupling 414.

FIGS. 6 and 7 also illustrate an example of the coupling 414 that has a proximal end 416 and a distal end 418. The material used to form the coupling 414 can be any suitable polymer that is compatible with the material of the large diameter and small diameter catheter segments. Therefore, the material used will differ based on the material of customer's requested large diameter shaft 402 and small diameter shaft 408. As described above, the proximal end 416 of the coupling 414 is molded such that a thin film of the material used to form the coupling 414 coats an interior surface 420 of the large diameter catheter 402. Therefore, a segment 422 of the proximal end 416 of the coupling 414 extends into the lumen 424 of the large diameter tube 402. Additionally, the mold described above can be configured such that a thin film of material used to form the coupling 414 coats an exterior surface 426 of the proximal end 410 of the small diameter shaft 408. Therefore, a segment 428 of the distal end 418 of the coupling 414 extends over the proximal end 410 of the small diameter shaft 408.

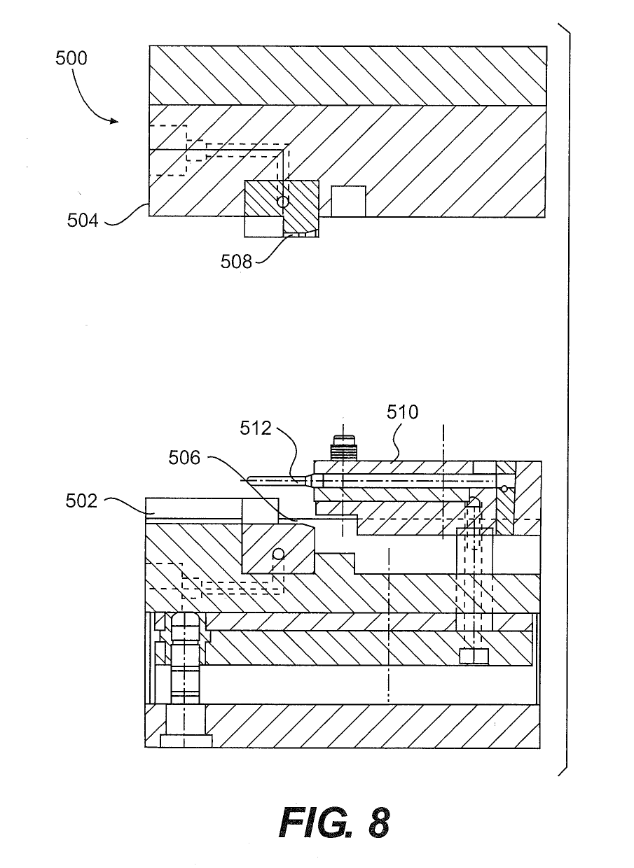

FIG. 8 illustrates one example of a mold 500 in accordance with the invention. The mold 500, illustrated in FIG. 8, includes a bottom plate 502 and a top plate 504. The bottom plate 502 of the mold 500 includes a first structure or insert 506 defining one side of an outside surface of the coupling or joint section that connects the large diameter shaft with the small diameter shaft of the catheter. The top plate 504 of the mold also includes a second structure or insert 508 defining another side of the outside surface of the coupling. The bottom plate 502 and the top plate 504 are also configured to accommodate the clam shell assembly 510 and the portion of the core pins 512 protruding from the end of the clam shell 510. In operation, the bottom plate 502 and the top plate 504 of the clam shell are compressed together, and the material being used to form the connection between the large diameter shaft and the small diameter shaft is injected into the mold 500. In some embodiments of the invention, the material is injected into the mold 500 using standard injection techniques.

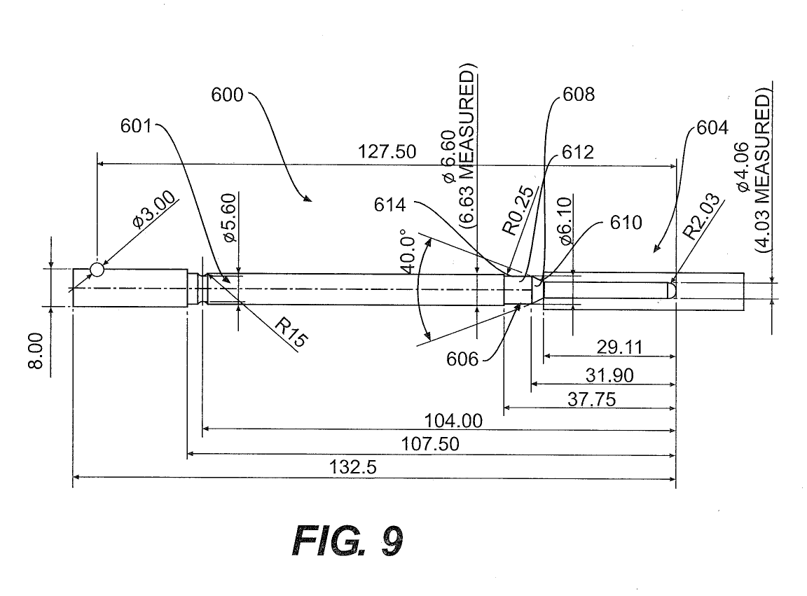

FIG. 9 illustrates one example of a core pin assembly 600 in accordance with the invention. FIG. 9 also includes an example set of possible dimensions for the core pin assembly 600 in accordance with an embodiment of the invention. It should, however, be noted that these dimensions are only one example of many different combinations of dimensions for the core pin assembly 600. The core pin assembly 600 can therefore have any dimensions as dictated by the diameter of the large diameter shaft and the diameter of the small diameter shaft as well as a particular profile of the coupling therebetween. The core pin assembly 600 includes a proximal end 602 and a distal end 604. The large diameter shaft can fit over the proximal end 602 of the core pin assembly 600. The small diameter shaft can fit over the distal end 604 of the core pin assembly 600. A middle section 606 of the core pin assembly 600 lies between the proximal end 602 and the distal end 604. The middle section 606 includes the flare 608 to form the coupling transition between the large diameter shaft and the small diameter shaft. As illustrated in FIG. 9, the diameter of the flare 608 increases from the distal end of the flare 610 to the proximal end of the flare 612. Additionally, the core pin assembly 600 includes a slight indentation 614 to allow a thin layer of the material being used to form the connector between the large diameter shaft and the small diameter shaft to coat an interior surface of a distal portion of the large diameter shaft.

Other embodiments of the invention can include using interlocking expandable core pins combined with injection molding in order to connect the catheter tubes. In some embodiments, the injection molding can be done layer by layer to form the transition between the tubes having different diameters. Over-molding can also be used to form the connector between the tubes having different diameters. Using these methods or the method described in more detail above, can allow for tubing having different inner diameters to be joined, especially where the ratio between the inner diameter of the tubing is large.

The many features and advantages of the invention are apparent from the detailed specification, and thus, it is intended by the appended claims to cover all such features and advantages of the invention which fall within the true spirit and scope of the invention. Further, because numerous modifications and variations will readily occur to those skilled in the art, it is not desired to limit the invention to the exact construction and operation illustrated and described, and accordingly, all suitable modifications and equivalents may be resorted to falling within the scope of the invention. In particular, the various elements of each embodiment discussed herein may be interchangeably used in other embodiments.

What is claimed is:

1. A method of forming a medical catheter tube having sections of different diameter, comprising:
providing first and second tubes having larger and smaller diameters, respectively;
positioning the first and second tubes onto a core pin assembly having an outer surface at one section which forms a contoured inner profile for a joint section to join the first and second tubes;
injection molding the joint section to join the first and second tubes, to form the catheter tube with the contoured inner profile at the joint section.

2. The method of claim 1, wherein the injection molding of the joint section comprises a plurality of injection molding steps to form a plurality of layers of the joint section.

3. The method of claim 1, flowing molten material over portions of the first and second tubes to form part of the joint section.

4. A method of forming a medical catheter tube having sections of different diameter coupled at a joint section, comprising:
providing a first length of tubing having a first, larger diameter;
providing a second length of tubing having a second, smaller diameter;
providing a set of one or more core pins arranged to provide a pre-determined inner surface profile of the catheter tube at the joint section;
positioning the first length of tubing over the set of core pins, to form a first sub-assembly;
placing the first sub-assembly into a clamshell fixture which holds the first length of tubing fixed to the set of core pins and leaves a first end portion of the set of core pins exposed, to form a second sub-assembly;
placing the second sub-assembly into a mold base;
positioning the second length of tubing over the first end portion of the set of core pins, leaving a section of the set of core pins exposed;
placing a cover mold portion over the mold base to form an inner space around the exposed section of the set of core pins; and
injection molding the joint section of the catheter tube by injecting a material into the inner space, said joint section coupling the first and second lengths of tubing and providing the pre-determined inner surface profile of the catheter tube at the joint section.

5. The method of claim 4, further comprising:
detaching the cover mold portion from the mold base and removing a third sub-assembly which includes the finished catheter tube, clamshell fixture and set of core pins;
placing the third sub-assembly into a catheter removal fixture to hold the third sub-assembly;
removing the set of core pins from the third sub-assembly; and
opening the clamshell fixture to remove the finished catheter tube.

6. The method of claim 4, wherein the injection molding of the joint section of the catheter tube comprises a plurality of injection molding steps to form a plurality of layers of the joint section.

7. The method of any one of claim 4, further comprising increasing an amount of core pins in the set of core pins.

8. The method of any one of claim 4, further comprising interlocking a core pin with at least one other core pin.

9. A catheter comprising:
a first section having a first diameter,
a second section having a second diameter greater than the first diameter; and
a joint section connecting the first section and the second sections, the joint section having a tapered portion tapering the inner diameter of the molded section down from the first diameter to the second diameter and the joint section is molded to the first section and the second section.

10. The catheter of claim 9, wherein the first and second sections extend into the joint section.

11. The catheter of claim 9, wherein the catheter was fabricated by:
providing first and second tubes having larger and smaller diameters, respectively;
positioning the first and second tubes onto a core pin assembly having an outer surface at one section which forms a contoured inner profile for a joint section to join the first and second tubes;
injection molding the joint section to join the first and second tubes, to form the catheter tube with the contoured inner profile at the joint section.

12. The catheter of claim 9, wherein the catheter was fabricated by:
providing a first length of tubing having a first, larger diameter;
providing a second length of tubing having a second, smaller diameter;
providing a set of one or more core pins arranged to provide a pre-determined inner surface profile of the catheter tube at the joint section;
positioning the first length of tubing over the set of core pins, to form a first sub-assembly;
placing the first sub-assembly into a clamshell fixture which holds the first length of tubing fixed to the set of core pins and leaves a first end portion of the set of core pins exposed, to form a second sub-assembly;
placing the second sub-assembly into a mold base;
positioning the second length of tubing over the first end portion of the set of core pins, leaving a section of the set of core pins exposed;
placing a cover mold portion over the mold base to form an inner space around the exposed section of the set of core pins; and
injection molding the joint section of the catheter tube by injecting a material into the inner space, said joint section coupling the first and second lengths of tubing and providing the pre-determined inner surface profile of the catheter tube at the joint section.

13. The catheter of claim 9, wherein the first and second sections were cut to a desired length prior to being attached to the joint section.

14. The catheter of claim 9, wherein when the joint section is molded molten material flows to cover at least part of an outer diameter of the first section and an outer diameter of the second section.

15. The catheter of claim 9, wherein the joint section is unitized to the first and second sections.
